(19) United States
(12) Patent Application Publication
(10) Pub. No.: US 2020/0190470 A1
(43) Pub. Date: Jun. 18, 2020

(54) TEMOZOLOMIDE RESISTANT CELLS AND AN INTEGRATED METHOD FOR CHARACTERIZING THE SAME

(71) Applicant: Council of Scientific & Industrial Research, New Delhi (IN)

(72) Inventors: Anu Raghunathan, Pune (IN); Selva Rupa Christinal Immanuel, Pune (IN)

(21) Appl. No.: 16/218,958

(22) Filed: Dec. 13, 2018

(51) Int. Cl. C12N 5/079; G01N 33/50; C12Q 1/6886; G16B 25/30

(52) U.S. Cl. CPC C12N 5/0618; G01N 33/5038; G01N 33/5058; G01N 30/7233; G16B 25/30; C12N 2506/30; C12Q 1/6886

(57) ABSTRACT

The present invention relates to temozolomide resistant glioblastoma cells lines. Further, the present invention relates to a method for identifying, screening and characterizing temozolomide resistant cells derived from glioblastoma cells lines in patients diagnosed with glioblastoma and undergoing treatment with temozolomide and/or in patients on the part to recovery to avoid or treat relapse. The issue of temozolomide resistance at the level of diagnosis and treatment of glioblastoma is undertaken by the present invention to be solved by the present invention.

### TECHNICAL FIELD OF THE INVENTION

The present invention relates to temozolomide resistant glioblastoma cells lines. More particularly, the present invention relates to a method for characterizing temozolomide resistant cells derived from glioblastoma cells lines in patients diagnosed with glioblastoma and undergoing treatment with temozolomide and/or in patients on the part to recovery to avoid and treat relapse.

### BACKGROUND AND PRIOR ART OF THE INVENTION

Glioblastoma multiforme (GBM) is a devastating form of brain cancer with a dismal median survival time, a high level of resistance to current therapy and common recurrence after treatment. The current standard therapy for GBM includes maximum debulking surgery, radiation and treatment with the monofunctional alkylating agent temozolomide (TMZ). Temozolomide (TMZ), an anti-cancer prodrug of Temodar® is an oral alkylating agent that is used in the treatment of glioblastoma multiforme (GBM) and astrocytomas. Temozolomide is an imidazotetrazine that has increased the prognosis of highly aggressive GBM, however, at least 50% of TMZ treated patients do not respond to TMZ.

Primarily, this is due to the over-expression of O⁶-methylguanine methyltransferase (MGMT) and/or lack of a DNA repair pathway in GBM cells. In the absence of MGMT repair, O⁶-methylguanine is suggested to initiate a futile cycle of mismatch repair (MMR) or alternately to trigger ataxia telangiectasia and Rad3 related protein kinase (ATR) activation through the action of several MMR proteins, leading to apoptosis and cell death.

Much of the resistance to TMZ observed clinically is due to high expression of MGMT (and subsequent repair of the lesion) or loss of MMR (therefore preventing the initiation of apoptotic signalling). Additionally, almost all patients eventually recur with the disease and the large majority of recurrent tumors are resistant to chemotherapy. There are currently few alternate treatment options for patients with TMZ resistant tumors and adjuvant chemotherapy options are an area of intense research.

The following GBM cells were reported consistently to be TMZ resistant: LN-18, T98G, UL38, CCF-STTG1 and U343-MG GBM cells.

The existence of small minority populations with differential histology and dye efflux properties within cancer cell lines has been known for decades; however, the underlying biochemical physiology of how this shapes functional drug response, susceptibility and resistance is still incompletely understood.

There is a need in the an to identify and characterize temozolomide resistant cancer cells in terms of drug efflux, growth/proliferation, nutrient preferences and metabolite profiling to drug dose response, thereby providing a tool for diagnosis and treatment of temozolomide resistance of glioblastoma cancer cells and its relapse.

### OBJECT OF THE INVENTION

It is an object of the present invention to identify and characterize temozolomide resistant cancer cells derived from a parental glioblastoma cell line, so that the physiological contributors to drug resistance in cancer cells can be identified.

It is another object of the present invention to provide a method for screening temozolomide resistant cancer cells in patients diagnosed with glioblastoma and undergoing treatment with temozolomide and/or in patients on the part to recovery to avoid relapse.

### SUMMARY OF THE INVENTION

In an aspect, the present invention provides temozolomide resistant neurospheroidal cells (NSP) derived from glioblastoma cells having the following characteristics:
(a) small spheroidal cells forming floating aggregates,
(b) differentiating ability to form glial cell types,
(c) a higher IC₅₀ value ranging from 900 μM to 1100 μM compared to parental glioblastoma cell lines having IC₅₀ value ranging from 700 μM to 800 μM,
(d) secreting alanine, glutamate and omithine by NSP cells, glycine and glutamate were secreted initially in fast dividing cells and then taken-up later suggesting a metabolic functional role in NSP, and
(e) glutamate and glutamine are sole sources of carbon/nitrogen for growth in NSP.

In another aspect, the present invention provides a method for screening temozolomide resistant neurospheroidal (NSP) cells from glioblastoma cell lines, the said method comprising;
(a) determining the differential growth kinetics of NSP cells by estimating the growth rates of neurospheroidal (NSP) cells;
(b) estimating the dose response parameters of NSP cells by estimating the 2-fold resistance indicated by higher IC₅₀, Eₘₐₓ and AUC values compared to the glioblastoma cell lines;
(c) metabolite profiling by liquid chromatography to indicate the utilization of amino acids Tyr, Trp, Val, Thr, Ser, Pro, Met, Phe, Lys, Leu/Ile, His in NSP cells somewhere between 120 and 168 h after culture; and
(d) performing phenotypic micro-array analysis to identify glutamate and glutamine as sole sources of carbon/nitrogen to facilitate growth in NSP's.

In yet another aspect, the present invention provides a kit for identification and characterization of temozolomide resistant cells lines in patients diagnosed with glioblastoma and undergoing treatment with temozolomide, the said kit comprising temozolomide resistance cell lines derived from U87MG (Grade-IV GBM cell line) along with instructions crucial for identifying, screening and characterization of the said cells, wherein the said instructions comprise;
(a) determining the differential growth kinetics of NSP cells by estimating the growth rates of cells, wherein the NSP growth rate is 30% lower than parental glioblastoma cell lines;
(b) estimating the dose response parameters of NSP cells, wherein a 2-fold resistance in the said NSP cells is indicated by higher IC₅₀, Eₘₐₓ and AUC values compared to glioblastoma cell lines;
(c) in-vitro differentiation of NSP cells indicating differentiation of the said cells into glial cell types;
(d) metabolite profiling by liquid chromatography to indicate the utilization of amino acids Tyr, Trp, Val, Thr, Ser, Pro, Met, Phe, Lys, Leu/Ile, His in NSP cells somewhere between 120 h and 168 h after culture;
(e) real-time PCR analysis determining the expression of CAN genes and ABC transporters;
(f) performing phenotype micro-array analysis to identify glutamate and glutamine as sole sources of carbon/nitrogen that facilitate growth in NSP's.

### DETAILED DESCRIPTION OF THE DRAWINGS

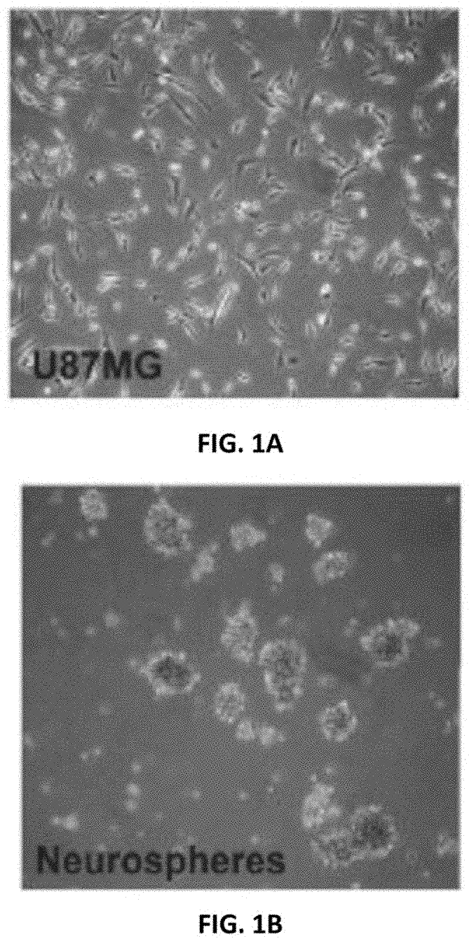

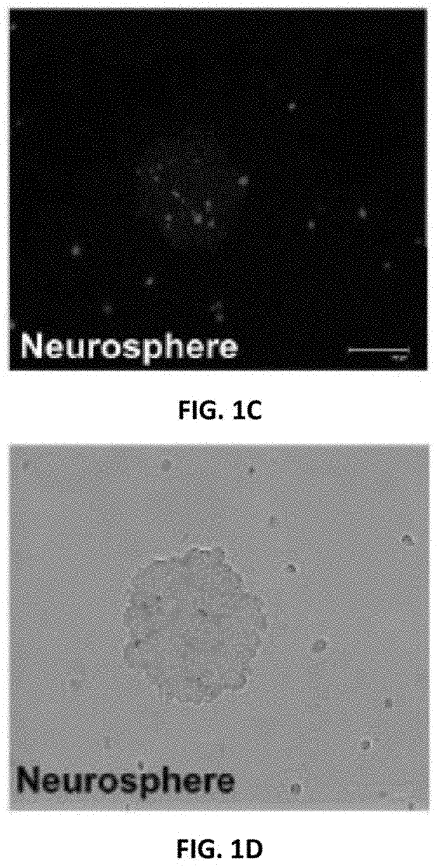

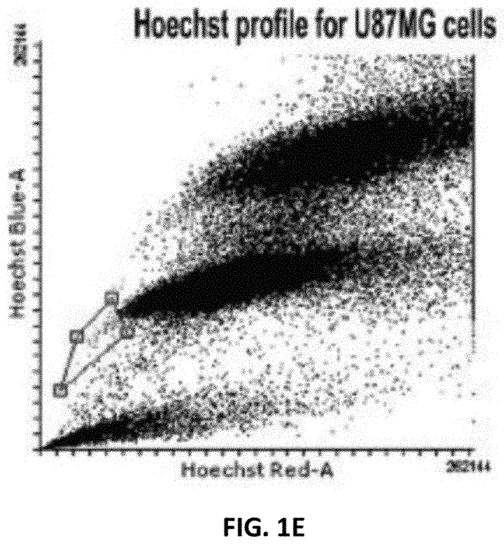

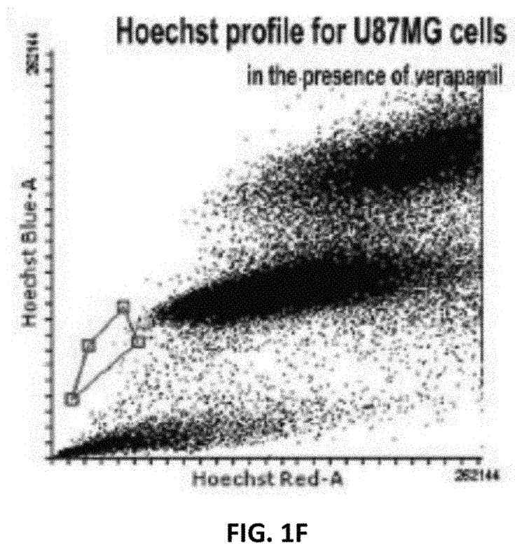

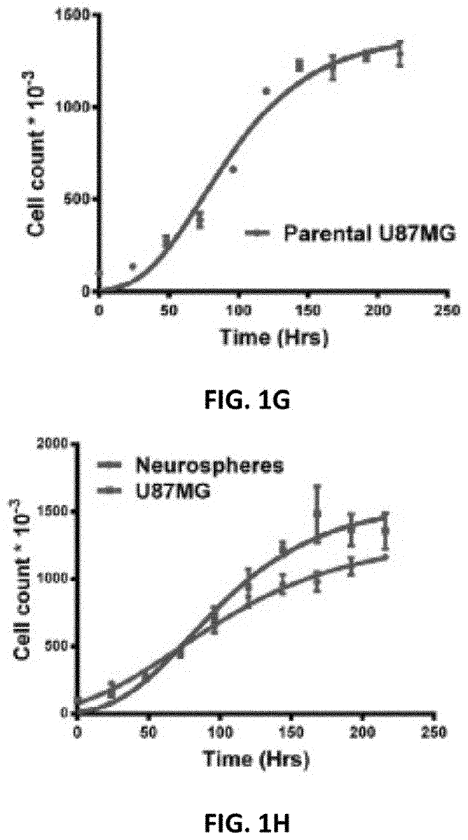

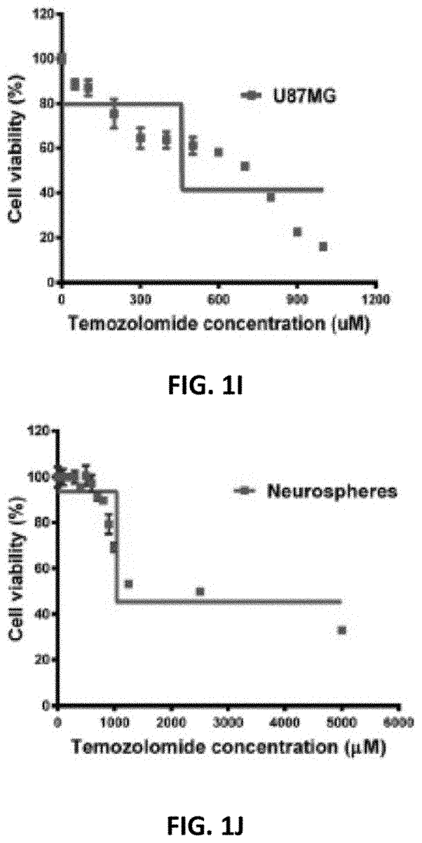

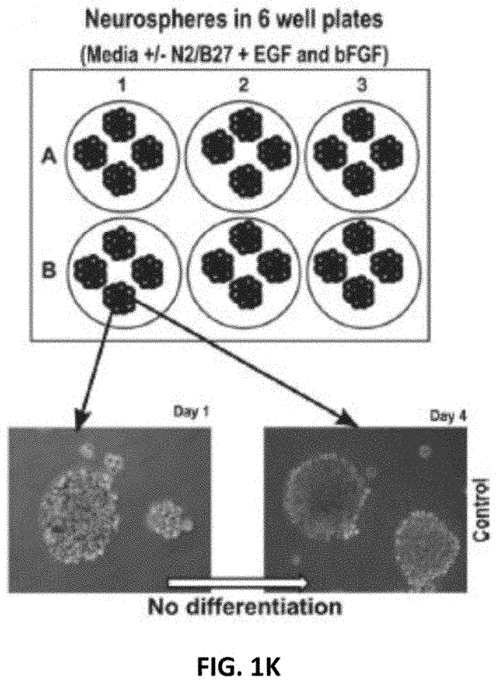

FIG. 1A depicts functional Characterization of U87MG and NSP, specifically, the morphological features of epithelial and adherent U87MG.

FIG. 1B depicts functional Characterization of U87MG and NSP, specifically the spherical suspension of NSP.

FIG. 1C depicts functional Characterization of U87MG and NSP, specifically with differential blue fluorescence intensity of Hoechst 33342 uptake.

FIG. 1D depicts functional Characterization of U87MG and NSP, specifically the merged bright-field light microscopy.

FIG. 1E depicts functional Characterization of U87MG and NSP, specifically the Hoechst profile of U87MG cells in the absence of verapamil wherein NSP cells were recognized as a small tail (red cells in polyhedron) extending first on the left side of GO/GI phase cells towards the lower “Hoechst Blue” signal.

FIG. 1F depicts functional Characterization of U87MG and NSP, specifically the gated side-population decreases in cell number in the presence of Verapamil confirming the blocking of dye efflux in NSP.

FIG. 1G depicts functional Characterization of U87MG and NSP, specifically the varying growth profiles fitted to Gompertz kinetic model of Parental U87MG.

FIG. 1H depicts functional Characterization of U87MG and NSP, specifically the separated NSP and U87MG.

FIG. 1I depicts functional Characterization of U87MG and NSP, specifically the dose response of U87MG showing IC50 of 745.6 mM.

FIG. 1J depicts functional Characterization of U87MG and NSP, specifically while that of NSP showing a 40% increase in IC50 1039 mM using non-linear regression.

FIG. 1K depicts functional Characterization of U87MG and NSP, specifically the Experimental setup: 6 well plate containing the NSP cells in media supplemented with N2 supplement added growth factors, bFGF and EGF/B27 supplement. Column 1 is the control media without N2/B27 supplement added growth factors, and the close-up shows no differentiation in the control.

FIG. 1L depicts functional Characterization of U87MG and NSP, specifically while confirming capability of NSP to differentiate on day 1 (24 h after seeding) and day 4.

FIG. 1M depicts functional Characterization of U87MG and NSP, specifically while confirming capability of NSP to differentiate on day 1 (24 h after seeding) and day 4. All microscopic images are captured using an EVOS® FLoid® system, ThermoFisher Scientific at magnification of 20× and 1×.

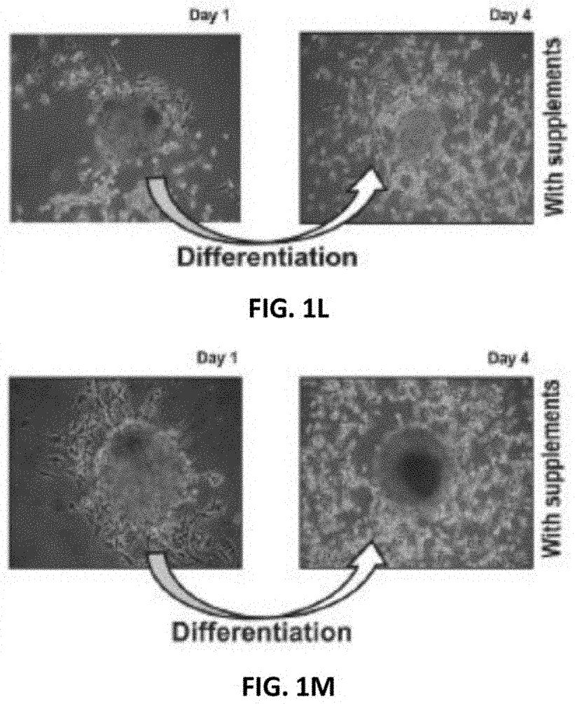

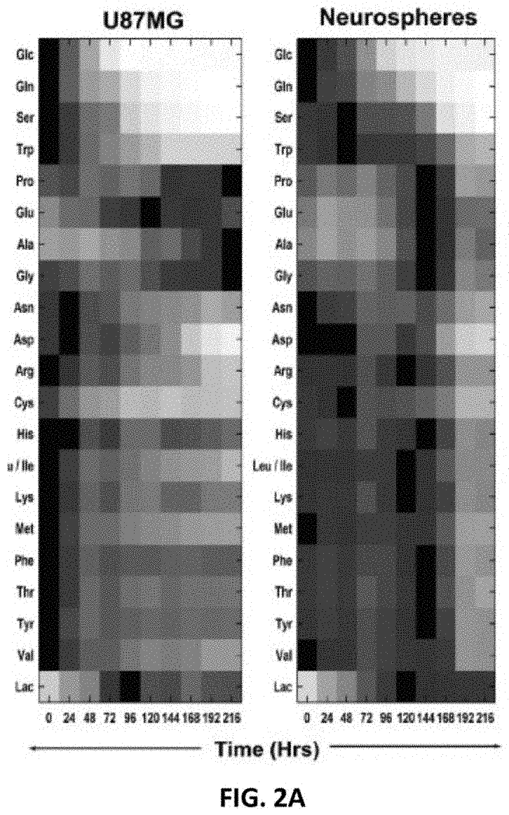

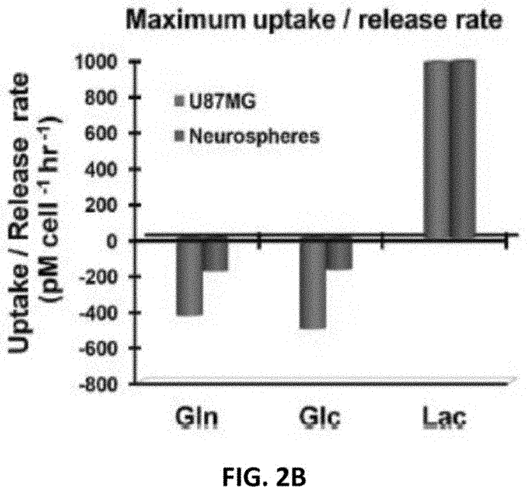

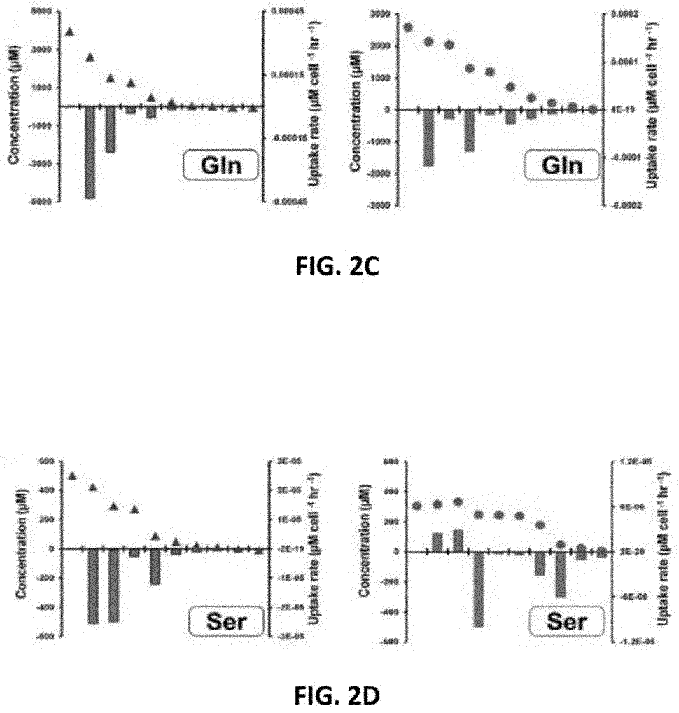

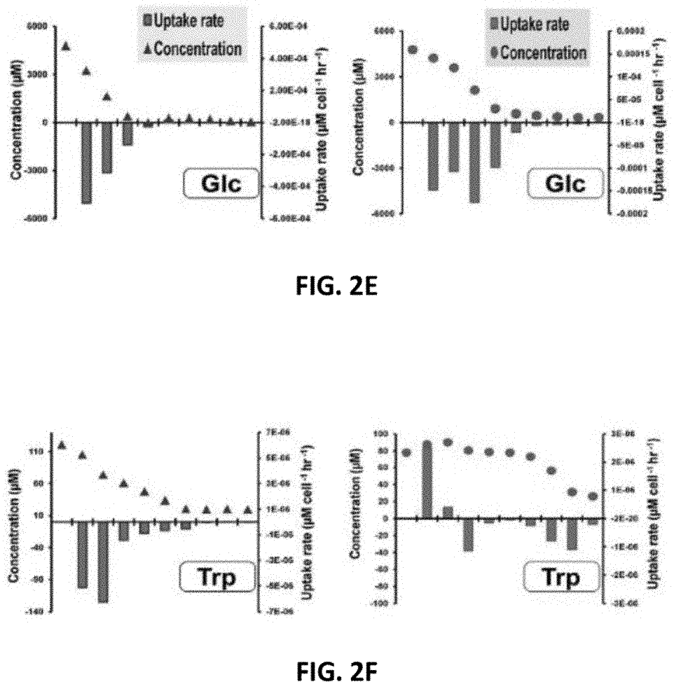

FIG. 2A depicts differential metabolite uptake and relate, specifically a heat map indicates the consumption/release of the amino acids in the extracellular medium over time. The profile varies across a scale of 0-1 wherein 1 corresponds to release/accumulation and 0 corresponds to the least value/uptake.

FIG. 2B depicts differential metabolite uptake and relate, specifically a maximum uptake/release rate of Gln, Glc and Lac. Each bar represents the slope from a linear fit of n ¼ 3 replicates ±SE.

FIG. 2C depicts differential metabolite uptake and relate, specifically a proliferating cells of U87MG/NSP in culture consumed glutamine in excess of other nutrients.

FIG. 2D depicts differential metabolite uptake and relate, specifically proliferating cells of U87MG/NSP in culture consumed serine in excess of other nutrients.

FIG. 2E depicts differential metabolite uptake and relate, specifically a proliferating cells of U87MG/NSP in culture consumed glucose in excess of other nutrients.

FIG. 2F depicts differential metabolite uptake and release, specifically a proliferating cells of U87MG/NSP in culture consumed while only U87MG consumed tryptophan in excess. NSP cells were found not to utilize tryptophan till 144 h (6 days) of growth. Uptake of glucose was faster in U87MG and slower in NSP. Standard three-letter abbreviations are used for amino acids. Glc-glucose; Lac-lactate.

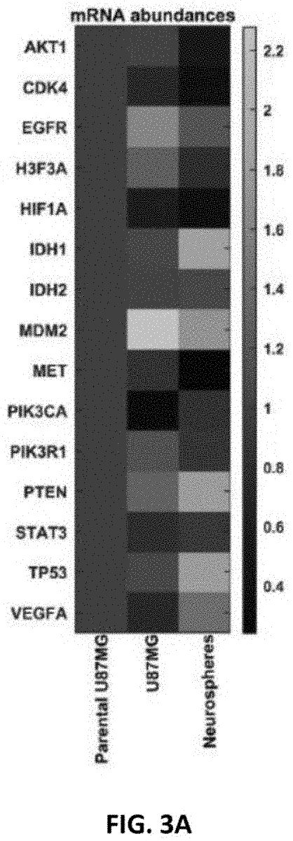

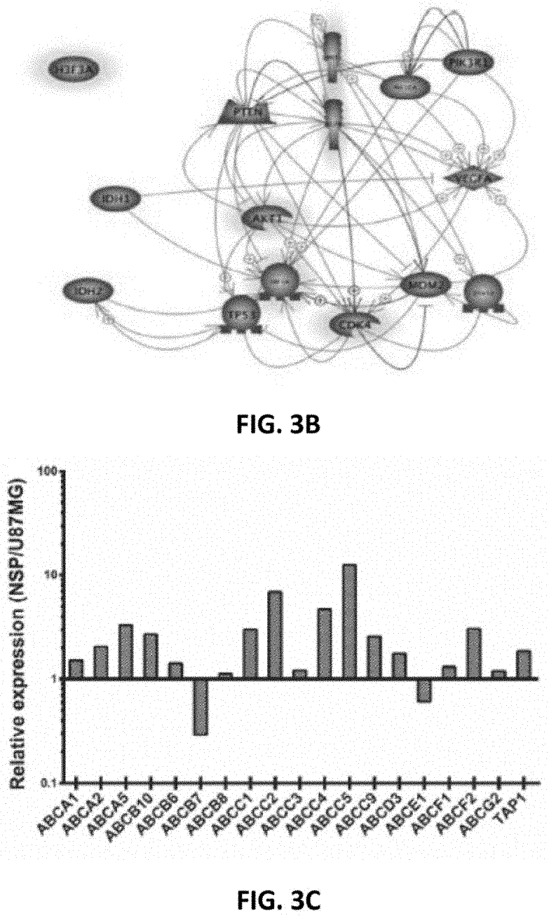

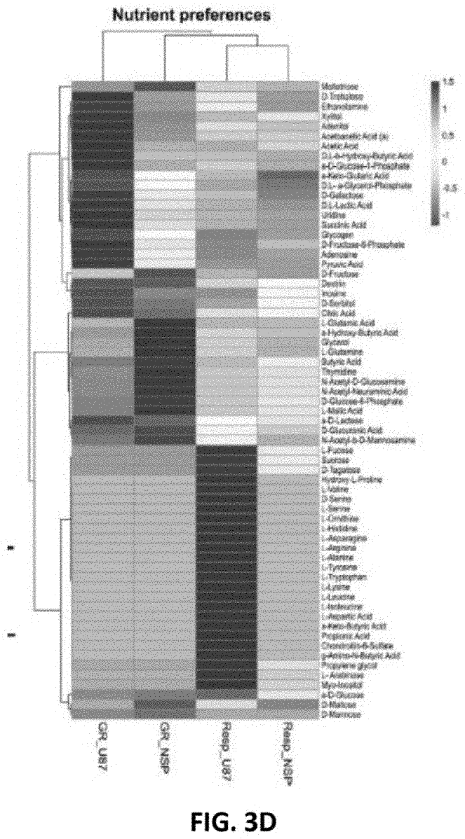

FIG. 3A depicts phenotypic plasticity in nutrient preferences and mRNA abundances, specifically a heat map showing the differential mRNA abundances of Receptor tyrosine kinases controlling metabolism and epigenetic control of methylation.

FIG. 3B depicts phenotypic plasticity in nutrient preferences and mRNA abundances, specifically a network of interactions between the differentially expressed (highlighted) CAN genes predicted using Pathway Studio 11.0.5.

FIG. 3C depicts phenotypic plasticity in nutrient preferences and mRNA abundances, specifically a differential mRNA abundances (NSP/U87MG) of 19 ABC transporters.

FIG. 3D depicts phenotypic plasticity in nutrient preferences and mRNA abundances, specifically a ABCB7 and ABCE1 control nutrient uptake while ABCG2 drug efflux Clustergram and heat map of nutrient preferences profiled using BIOLOG phenotypic microarrays. Euclidean clustering differentially correlates growth (GR) and respirations (Resp) rates.

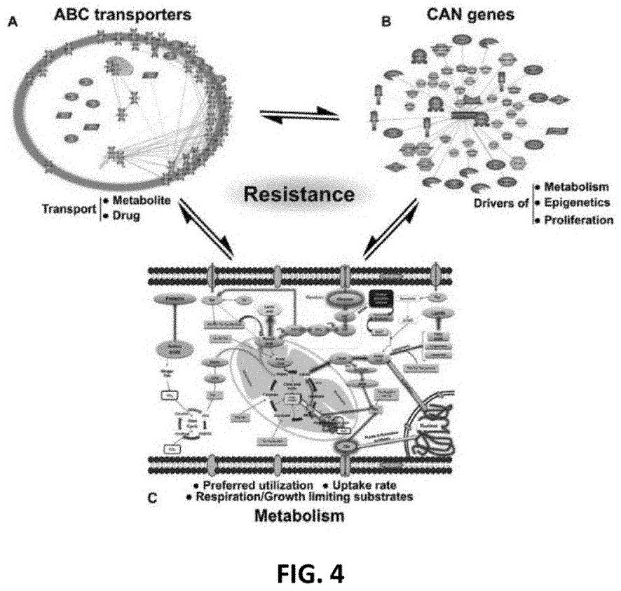

FIG. 4 depicts the Integrative paradigm for Temozolomide resistance. The contributions in part by metabolism, mRNA abundances of ABC transporters and CAN genes to TMZ resistance in NSP. Representation of (A) the localization of ABC transporters in the cell (B) network analysis of 23 CAN genes using Pathway Studio™ and (C) potential metabolic rewiring indicated by red (NSP) and blue (U87MG) arrows

### DETAILED DESCRIPTION OF THE INVENTION

The invention will now be described in detail in connection with certain preferred and optional embodiments, so that various aspects thereof may be more fully understood and appreciated.

In a preferred embodiment, the present invention provides temozolomide resistant neurospheroidal (NSP) cells derived from glioblastoma cell lines having the following characteristics:
(a) small spheroidal cells forming floating aggregates,
(b) differentiating ability to form glial cell types,
(c) a higher IC₅₀ value ranging from 900 μM to 1100 μM compared to parental glioblastoma cell lines having IC₅₀ value ranging from 700 μM to 800 μM,
(d) secreting alanin, glutamate and ornithine by NSP cells, glycine and glutamate were secreted initially in fast dividing cells and then taken-up later suggesting a metabolic functional role in NSP, and
(e) glutamate and glutamine are sole sources of carbon/nitrogen for growth in NSP.

In accordance with the aforesaid embodiment, the present invention provides neurospheres (NSP) as small spheroidal cells forming floating aggregates (FIG. 1B). Further, differential fluorescent intensities characterized NSP from parental glioblastoma cell lines in the heterogeneous population (FIGS. 1C and D).

As regards in-vitro growth differentiation of the NSP cells vis-à-vis the parental glioblastoma cell line, the present invention provides the NSP growth rate is 30% lower than the parental glioblastoma cell line and thus the maximum population density achieved at the end of 216 h was higher for U87MG as compared to NSP.

As regards Temozolomide dose response curves, the present invention provides a higher IC₅₀ for NSP (1039 mM) indicating a potential lower efficacy of the drug on these cells (FIG. 1J). The fold resistance was calculated to be 40%. The Eₘₐₓ values for NSP are almost three fold higher than that of glioblastoma cell line indicating higher efficacy of TMZ on U87MG as compared to NSP (Table 2).

As regards the IC₅₀ values of NSP cells differentiated into glial cell types, these differentiated cells of NSP showed a TMZ dose response in between that of NSP and U87MG, exhibiting an IC₅₀ value of 817.5 mM and growth rate of 0.01634 hr⁻¹.

As regards, the metabolite profiling in NSP cells and the parental cell line, it was observed in the present invention that NSP only secreted alanine and glutamate. Glycine and glutamate were secreted initially in fast dividing cells and then taken-up later suggesting a metabolic functional role in NSP.

Another feature distinct from the parental cell line, i.e. U87MG was that NSP however seems to show maximum uptake of these amino acids after a 24 h lag. The utilization of the amino acids Tyr, Trp, Val, Thr, Ser, Pro, Met, Phe, Lys, Leu/Ile, His in NSP cells is somewhere between 120 and 168 h that continued until growth was monitored to 216 h (FIG. 2A). This indicated the demand for these nutrients may exceed the endogenous synthetic capacity in NSP and thus necessitate uptake during that time period.

In another preferred embodiment, the present invention provides a method for screening temozolomide resistant neurospheroidal (NSP) cells from glioblastoma cell lines, the said method comprising;
(a) determining the differential growth kinetics of NSP cells by estimating the growth rates of cells, wherein the NSP growth rate is 30% lower than parental glioblastoma cell lines;
(b) estimating the dose response parameters of NSP cells, wherein a 2-fold resistance in the said NSP cells is indicated by higher IC₅₀, Eₘₐₓ and AUC values compared to glioblastoma cell lines;
(c) in-vitro differentiation of NSP cells indicating differentiation of the said cells into glial cell types;
(d) metabolite profiling by liquid chromatography to indicate the utilization of amino acids Tyr, Trp, Val, Thr, Ser, Pro, Met, Phe, Lys, Leu/Ile, His in NSP cells somewhere between 120 h and 168 h after culture;
(e) real-time PCR analysis for determining CAN genes and ABC transporters expression;
(f) performing phenotype micro-array analysis to identify glutamate and glutamine as sole sources of carbon/nitrogen that facilitate growth in NSP's.

In a further embodiment, the present invention provides Temozolomide to be potentially efficacious in controlling the NSP growth but only at concentrations that are 40% higher than the IC50 value ranging from 1000 μM to 1100 μM.

In another embodiment, the present invention provides that TMZ resistance is in part due to the effect of specific receptor tyrosine kinases and substrate ABC transporters that rewire metabolism through preferential utilization.

A higher level of cytokine STAT3 in NSP potentially turns on the MYC transcriptional program and induces preferential consumption of glutamine. The simultaneous negative regulation of ACL by AKT1 potentially lowers the acetyl CoA pool and hence the acetate available for histone acetylation. A reduced aerobic glycolytic flux reflective of higher NAD levels may control SIRTUIN family transcriptional repressors and silence chromatin via decreasing histone acetylation. This partially explains differential tryptophan metabolism (growth limiting U87MG) and suggests a role for picolinate carboxylase in the balance between NAD derived nucleotide synthesis and acetyl-CoA driven acetylation. Higher levels of AKG (TCA pool) also indicate epigenetic connections to the Jumonji family of histone de-methylases that could prevent methylation of histones. This may explain the varied dose response relations and increased IC50 for TMZ. The dependency on glucose and pyruvate, and not glutamine for growth/respiration also suggests normal levels of methylation in U87MG. Similar MGMT mRNA levels suggest functional DNA repair in the absence of TMZ. The role of AKG in demethylation of DNA and its effects on drug sensitivity/resistance needs further testing.

In a further embodiment, the present invention provides a kit for identification and characterization of temozolomide resistant cells lines in patients diagnosed with glioblastoma and undergoing treatment with temozolomide, the said kit comprising temozolomide resistance cell lines derived from U87MG (Grade-IV GBM cell line) along with instructions crucial for screening and characterization the said cells, wherein the said instructions comprise;
(a) determining the differential growth kinetics of NSP cells by estimating the growth rates of cells, wherein the NSP growth rate is 30% lower than parental glioblastoma cell lines;
(b) estimating the dose response parameters of NSP cells, wherein a 2-fold resistance in the said NSP cells is indicated by higher IC₅₀, Eₘₐₓ and AUC values compared to glioblastoma cell lines;
(c) in-vitro differentiation of NSP cells indicating differentiation of the said cells into glial cell types;
(d) metabolite profiling by liquid chromatography to indicate the utilization of amino acids Tyr, Trp, Val, Thr, Ser, Pro, Met, Phe, Lys, Leu/Ile, His in NSP cells somewhere between 120 h and 168 h after culture;
(e) real-time PCR analysis for identifying CAN genes and ABC transporters expression;
(f) performing phenotype micro-array analysis to identify glutamate and glutamine as sole sources of carbon/nitrogen that facilitate growth in NSP's.

### EXAMPLES

The following examples are given by way of illustration of the present invention and therefore should not be construed to limit the scope of the present invention.

### Example 1: Cell Culture

An authenticated U87MG cell line (HTB-14; Human Glioblastoma Multiforme from ATCC;) was cultured in conditions as per ATCC guidelines. Neurospheres (NSP) were maintained in neurobasal medium supplemented with B27 supplement, 0.2 mg/mL each of epidermal growth factor, EGF and basic fibroblast growth factor, (bFGF). NSP were cultured as floating spheres in low attachment T-75 flasks or 6 well/24 well plates (Nunc™). All chemicals and labware was purchased from ThermoFisher Scientific™.

The glioblastoma cell line U87MG contained a sub-population (0.1%) of Hoechst-effluxing cells. The sub-population (NSP) was confirmed with Verapamil, an ABC transporter L-type calcium channel blocker and inhibitor of dye efflux. The separated populations were tested for morphological and phenotypic heterogeneity and temozolomide dose response.

### Example 2: Fluorescence Microscopy and Flow Cytometry Based Separation of Cells

Hoechst 33342 stain (1 mg/mL) was used for all fluorescence studies on EVOS® FLoid® cell imaging system. The subpopulation sorting assay as previously described was performed for FACS with cells at 70-80% confluency using BD FACSAria III (BD biosciences Pvt. Ltd) and analysed using BD FACSDiva™ software v6.1.3. Cells were captured in a Hoechst Blue versus Hoechst Red dot plot in the presence and absence of Verapamil.

Under bright field microscopy, cultures of separated U87MG showed glial cell characteristics with epithelial cell morphology (FIG. 1A). Neurospheres (NSP) were small spheroidal cells forming floating aggregates (FIG. 1B). Differential fluorescent intensities characterized NSP from U87MG in the heterogeneous population (FIGS. 1C and D).

The multi-step gating strategy based on differential fluorescence profiles was critical for characterizing and sorting the subpopulation from the main population. The flow-cytometric profile based on the forward scatter (FSC, indicative of cell size) and side scatter (SSC, indicative of cell granularity) allowed distinguishing viable cells from cell debris. NSP cells were recognized as a dim tail extending first on the left side towards the lower “Hoechst Blue” signal (FIG. 1E) and confirmed using dye efflux through the Verapamil-sensitive ATP binding cassette (ABC) transporter, ABCG2 (FIG. 1F).

### Example 3: Growth/Proliferation Studies

Growth/proliferation of the cells (Parental U87MG, U87MG and NSP) was monitored via cell counts over a period of 216 h (9 days). The initial seeding (No) was ˜10000 cells per well. All cells were harvested every 24 h and counted using hemocytometer using trypan blue dye exclusion assay. NSP was trypsinized before counting. Growth curves were graphed and data fitted with Gompertz function (GraphPad Software, San Diego Calif. USA).

Growth profiles (FIGS. 1G and H) and parameters (Table 1) for the cell types monitored in the proliferation experiment were varied. The Gompertz function representing growth kinetics for both cell types was:

N(t)=No exp(ln(N(t)/No)[1−exp(−kt)]

where No defines the initial seeding density of the cells, Nt is the number of cells at time t, and k is the maximum specific growth constant (Table 1).

TABLE 1
Growth parameters determined based on Gompertz growth. The growth
rates of U87MG were [symbol] higher than that of NSP [symbol] to
a doubling time of [symbol] and [symbol] for U87MG and NSP respectively.

| | | [symbol] | | |
|---|---|---|---|---|
| [symbol] growth | [symbol] | (hr⁻¹) | | |
| parameters | (cell numbers) | Gompertz | [symbol] | [symbol] |
| Neurophores | 1.304 [symbol] 10 [symbol] | 0.01473 | 0.0 [symbol] | 0.9733 |
| [symbol] | 1. [symbol] 10 [symbol] | 0.01973 | 0.0219 | 0.9421 |
| Parental [symbol] | 1.383 [symbol] 10 [symbol] | 0.0 [symbol] | 0.0 [symbol] | 0. [symbol] |

[symbol]

indicates data missing or illegible when filed

The doubling times calculated from the growth rates (Table 1) were 35.12 and 47.05 h for U87MG and NSP respectively. The fitted Gompertz function (FIG. 1H) reaches its horizontal asymptote for U87MG and NSP after 6.17 and 4.8 doublings respectively. The maximum specific growth rates calculated were 0.014 and 0.02 hr⁻¹ for NSP and U87MG respectively. The NSP growth rate is 30% lower than U87MG and thus the maximum population density reached at the end of 216 h was higher for U87MG as compared to NSP. The parental population had a growth rate of 0.02 hr⁻¹ and doubling time of 36 h (FIG. 1G and Table 1).

### Example 4: Temozolomide Dose Response Curves

For dose-response experiments, four replicates at ˜20,000 cells per well were plated in 96-well Nunc™ tissue culture plates in full growth medium for 24 h, treated with different doses of TMZ in serial dilutions (0.05Me5M) followed by cell viability tests using the MTT assay. Appropriate cell controls (without TMZ treatment) were used to calculate IC₅₀.

The varied dose response and growth inhibition patterns of U87MG and NSP are observed in the steepness of the dose response curve for TMZ (Temodar) (FIGS. 1I and J), differences in maximum effect and the more classical drug potency measurements (IC₅₀ and Emax). The summary (Table 2) of IC50 values indicative of the potency of the drug, Emax values a measure of efficacy. Hill Slopes (HS) indicating effect per unit of drug and area under the curves reflected the overall response of the cell lines to TMZ. A lower IC50 value for U87MG cells (745.6 mM) indicates higher potency of TMZ against these cells (FIG. 1I). A higher IC50 for NSP (1039 mM) indicates a potential lower efficacy of the drug on these cells (FIG. 1J). The fold resistance was calculated to be 40%. The Emax values for NSP are almost three fold higher than that of U87MG indicating higher efficacy of TMZ on U87MG as compared to NSP (Table 2).

TABLE 2
Summary of TMZ dose response. An increase in IC₅₀ by 40% for
NSP indicates a potential lower efficacy of the drug. The
Emax and Hill slope values indicate higher efficacy of TMZ
on the main cell line as compared to the NSP, AUC, the combined
potency and efficacy of TMZ, was higher for NSP.

| Dose response summary | U87MG | Neurospheres |
|---|---|---|
| IC₅₀ (μM) | 745.6 | 1039 |
| Emax | 25.53 | 49.29 |
| Log₂ (Hill Slope) | −1.95904 | 0 |
| AUC | 53039 | 276836 |

However, a HS value of near 0 for NSP indicates a potentially higher efficacy, at least in culture. AUC combines potency and efficacy of a drug into a single parameter. AUC values were compared across U87MG and NSP exposed to the same range of TMZ concentrations indicate higher impact of the drug on U87MG. Temozolomide is potentially also shown to be efficacious in controlling the NSP growth but only at concentrations that are 40% higher (indicated by fold resistance ratio 1.41).

### Example 5: In-Vitro Differentiation of NSP

NSP were grown in DMEM followed by addition of 1% N2 supplement. Growth factors, bFGF and EGF reconstituted in 0.1% BSA solution (100 mg/mL) were used. An increased B27 (2%) supplemented after 3 days drove the differentiation. The differentiated cells (NDx) were cultured separately using the same method of culture of U87MG cells. The ability of NSP to undergo differentiation and form specialized cell types was confirmed in an assay with specific growth factors and supplements. The differentiated cells (NDx) exhibited glial morphology and were adherent in contrast to the NSP (spheroidal) population (FIG. 1K-N). These differentiated cells showed a TMZ dose response in between that of NSP and U87MG, exhibiting an IC₅₀ value of 817.5 μM and growth rate of 0.01634 hr⁻¹. The exo-metabolome analysis of growth media also showed similarities to U87MG especially with respect to glucose and glutamine consumption. Serine, tryptophan and glutamate profiles mimicked NSP.

### Example 6: Metabolite Profiling Using Liquid Chromatography—High Resolution Mass Spectrometry (LC-HRMS)

Samples harvested during growth every 24 h over a period of 9 days were used for metabolic profiling performed in Accela 1250 ultra-performance liquid chromatography (UPLC) in tandem with Thermo Q-exactive high resolution mass spectrometer (HRMS) using heated electrospray ionization (HESI) interface. Parameters of LC-HRMS run and sample extraction protocols and standard preparation are provided. Accurate mass-extracted ion chromatograms (AM-XIC) of various metabolites were generated followed by peak confirmation by MS/MS spectral peak matching. Confirmed metabolites were quantified using internal standard normalized linear regression models. The raw and processed data of MS/MS confirmations, concentration ranges and regression fits are provided.

Quantitative exo-metabolite profiling identifies differential dynamics of nutrient uptake in the NSP population. LC-MS/MS was used to monitor the differential nutrient uptake of glucose and amino acids required for growth (FIG. 2A). Glucose was taken up linearly by U87MG and correlated with release of lactate, consistent with Warburg effect in transformed cells. NSP had a slight lag before glucose uptake and was consumed exponentially at lower rates (FIG. 2). Among the amino acids, glutamine consumption (FIGS. 2B and C) was quantitatively the highest and closely mirrored glutamate secretion in media. The nutrients completely depleted during growth in both U87MG and NSP were glucose, glutamine and serine although with varied dynamics (FIG. 2C-F). Tryptophan was utilized completely by U87MG alone indicating different functional roles in NSP (FIG. 2F).

The maximum uptake rates of these amino acids in U87MG being highest in the first 24 h decreasing several fold by the end of 96 h. NSP however seems to show maximum uptake of these amino acids after a 24 h lag. The utilization patterns indicate a linear decrease of these nutrients for U87MG while during NSP growth, an exponential decrease of the same nutrients was observed with a 48 h lag corresponding to the end of the first doubling (FIG. 2A). Ala, Glu, Gly and Pro were secreted by U87MG, similar to glioblastoma legacy data.

NSP however only secreted alanine and glutamate. Glycine and glutamate were secreted initially in fast dividing cells and then taken-up later suggesting a metabolic functional role in NSP. Several amino acids (Cys, Leu/Ile, Lys, Gly, Met, Phy, Ser, Thr, Tyr and Val) were utilized linearly in the first 48 h of growth by U87MG. The same was observed in NSP only after the first doubling (FIG. 2A). Another feature distinct from U87MG was the utilization of the amino acids Tyr, Trp, Val, Thr, Ser, Pro, Met, Phe, Lys, Leu/Ile, His in NSP cells somewhere between 120 and 168 h that continued until growth was monitored to 216 h (FIG. 2A). This indicated the demand for these nutrients may exceed the endogenous synthetic capacity in NSP and thus necessitate uptake during that time period.

### Example 7

### (i) Selection of CAN Genes Using Pathway Studio™ Analysis:

Pathway Studio 11.0.5 software from Elsevier (https://product.pathwaystudio.com/mammalcedfx/) was used to select CAN genes in silico. Initial pathways/networks implicated in GBM were reconstructed using related key terms. Networks were validated (confidence score-3) and casual pathways inferred by filtering to include >25 references. Pathway containing 23 genes and 1 miRNA resulted. These genes were expression profiled to quantitate relative mRNA abundances.

### (ii) Real-Time PCR Analysis of CAN Genes:

All primers designed were based on the Primer3web (http://primer3.ut.ee/) solutions and validated using NCBI BLAST (Basic Local Alignment Search Tool) (http://www.ncbi.nlm.nih.gov/BLAST). Stable reference genes across samples were selected based on GeNorm analysis. GAPDH and RPL13A selected as control genes had average expression stability (M) of less than 0.4. For high specificity SYBR green and hydrolysis probe assays were performed. LNA hydrolysis probes were designed using the universal probe library assay design center from Roche Applied Science, Ltd. (http://www.universalprobelibrary.com). qPCR was run on a Light Cycler 480 instrument from Roche Applied Sciences following MIQE (Minimum Information for Publication of Quantitative Real-Time PCR Experiments) guidelines.

### (i) Real-Time PCR Analysis of ABC Transporters:

Real-Time quantitative PCR (RT-qPCR) was used to quantify gene expression using the 44 ABC transporter gene array (TaqMan Array Human ABC Transporter Panel) in duplicates with 4 housekeeping genes. Protocols were followed as per manufacturer instructions. Profiling was done using a Light Cycler 480 instrument (Roche Applied Sciences) and data analysis using LCS480 1.5.1.62 software.
   (iv) mRNA Abundances of CAN and ABC Transporters Genes
The choice of genes for qRT-PCR was based on Pathway Studio™ analysis. An interaction network of 23 genes) based on high confidence scores was generated and included Cancer Candidate (CAN) genes. Statistically significant variation in expression of CAN genes (FIG. 3A) between the parental U87MG, U87MG and NSP populations was observed relative to the reference genes. 16 CAN genes were found to be different in their mRNA abundances (FIG. 3A) and their interactions were predicted using pathway analysis (FIG. 3B).

The differential mRNA abundances of signalling pathway controllers like the receptor tyrosine kinases may shape metabolic dynamics while modulation in histone deacetylases and methyl transferases potentially contribute to changes observed in TMZ dose response relation (increasing IC50 values and decreasing sensitivity). It is well known that p53 and PTEN play a pivotal role in tumor suppression and both have less mRNA in U87MG with respect to NSP (FIG. 3A).

Higher mRNA of AKT1 in U87MG with respect to NSP is evident in the differential glucose and amino acid uptake patterns. IDH1/IDH2 has higher mRNA in NSP in comparison with U87MG indicating potential reprogramming of the pathways around AKG that could eventually cause downstream effects on the acetylation profiles. Increased PIK3CA (5 fold change) in NSP could potentially dictate changes involving phosphorylation governing differential nutrient uptake. VEGFA and MDM2 also have higher mRNA expressed in NSP compared to U87MG. Lower CDK4 mRNA abundances as compared to U87MG indicated a potential cell cycle arrest that needs to be overcome for NSP to proliferate. Lowered H3F3A mRNA in NSP indicates potential changes in histone acetylation patterns. The gene expression of 19 out of the 44 ATP-binding cassette (ABC) transporters profiled, varied between NSP and U87MG (FIG. 3C). The differential mRNA abundances (17 higher and 2 lower for NSP) potentially contribute to the efficiency of drug and nutrient metabolite transport and efflux. Relative expression highlighted ABCB7 and ABCE1 to be lower for NSP with respect to U87MG. ABCC5 had the highest variation and showed 10-fold increase in mRNA abundances. Maximum variation was seen in the ABCC sub family. ABCG2, linked to TMZ transport and Verapamil, showed 20% increase in mRNA in NSP cells. The varied abundance of transporters was also linked to metabolite transport in the network analysis and legacy data.

### Example 8: Phenotype Microarray Analysis

Biolog Phenotype Micro-Arrays™ PM-M1-M2 from Biolog, Inc. USA (www.biolog.com) were tested using protocols suggested by the manufacturer. All plates were incubated at 37° C. in CO₂ incubator and absorbance monitored at 590 nm at regular time intervals to calculate respirations rates. Phenotypic plasticity defined by nutrient preferences for growth and respiration. The cellular energetics and pathways involved in the metabolism of U87MG and NSP during the nutrient restricted state (only one carbon or nitrogen source) were addressed using Biolog Phenotype MicroArrays™ plates (PM-M and PM-M2). The varied metabolic fingerprints of respiration and growth on 73 carbon and nitrogen sources were analyzed using Euclidean clustering (FIG. 3D) and indicated coupled growth and respiration for U87MG. Glutamate and glutamine were identified as sole sources of C/N for growth in NSP while Glucose, AKG and Pyruvate supported growth in U87MG.

### ADVANTAGES OF THE PRESENT INVENTION

The present invention provides integrative paradigms across molecular hierarchies in the cell which helps to design combinatorial treatments using metabolite supplements to overcome drug resistance.

1. Temozolomide resistant neurospheroidal (NSP) cells derived from glioblastoma cell lines having the following characteristics:
(a) small spheroidal cells forming floating aggregates,
(b) differentiating ability to form glial cell types,
(c) a higher IC₅₀ value ranging from 900 μM to 1100 μM compared to parental glioblastoma cell lines having IC₅₀ value ranging from 700 μM to 800 μM,
(d) secreting alanine and glutamate by NSP cells, glycine and glutamate were secreted initially in fast dividing cells and then taken-up later suggesting a metabolic functional role in NSP, and
(e) glutamate and glutamine are sole sources of carbon/nitrogen for growth in NSP.

2. A method for screening temozolomide resistant neurospheroidal (NSP) cells from glioblastoma cell lines, the said method comprising;
(a) determining the differential growth kinetics of NSP cells by estimating the growth rates of cells, wherein the NSP growth rate is 30% lower than parental glioblastoma cell lines;
(b) estimating the dose response parameters of NSP cells, wherein a 2-fold resistance in the said NSP cells is indicated by higher IC₅₀, Eₘₐₓ and AUC values compared to glioblastoma cell lines;
(c) in-vitro differentiation of NSP cells indicating differentiation of the said cells into glial cell types;
(d) metabolite profiling by liquid chromatography to indicate the utilization of amino acids Tyr, Trp, Val, Thr, Ser, Pro, Met, Phe, Lys, Leu/Ile, His in NSP cells somewhere between 120 h and 168 h after culture;
(e) real-time PCR analysis for determining CAN genes and ABC transporters expression;
(f) performing phenotype micro-array analysis to identify glutamate and glutamine as sole sources of carbon/nitrogen that facilitate growth in NSP's.

3. The method for screening temozolomide resistant neurospheroidal (NSP) cells from glioblastoma cell lines as claimed in claim 2, wherein the NSP growth rate is 30% lower than the parental glioblastoma cell line.

4. The method for screening temozolomide resistant neurospheroidal (NSP) cells from glioblastoma cell lines as claimed in claim 2, wherein a higher IC50 for NSP ranging from 1000 μM to 1100 μM indicates a potential lower efficacy of the drug on NSP cells.

5. The method for screening temozolomide resistant neurospheroidal (NSP) cells from glioblastoma cell lines as claimed in claim 2, wherein the Eₘₐₓ values for NSP are almost three fold higher than that of parental glioblastoma cell lines indicating higher efficacy of TMZ on U87MG as compared to NSP.

6. A kit for identification and characterization of temozolomide resistant cells lines in patients diagnosed with glioblastoma and undergoing treatment with temozolomide, the said kit comprising temozolomide resistance cell lines derived from U87MG (Grade-IV GBM cell line) along with instructions crucial for screening and characterization the said cells, wherein the said instructions comprise;
(a) determining the differential growth kinetics of NSP cells by estimating the growth rates of cells, wherein the NSP growth rate is 30% lower than parental glioblastoma cell lines;
(b) estimating the dose response parameters of NSP cells, wherein a 2-fold resistance in the said NSP cells is indicated by higher IC₅₀, Eₘₐₓ and AUC values compared to glioblastoma cell lines;
(c) in-vitro differentiation of NSP cells indicating differentiation of the said cells into glial cell types;
(d) metabolite profiling by liquid chromatography to indicate the utilization of amino acids Tyr, Trp, Val, Thr, Ser, Pro, Met, Phe, Lys, Leu/Ile, His in NSP cells somewhere between 120 h and 168 h after culture;
(e) real-time PCR analysis for identifying CAN genes and ABC transporters expression;
(f) performing phenotype micro-array analysis to identify glutamate and glutamine as sole sources of carbon/nitrogen that facilitate growth in NSP's.
